(19) United States
(12) Patent Application Publication
(10) Pub. No.: US 2018/0070896 A1
(43) Pub. Date: Mar. 15, 2018

(54) INTRAORAL SENSOR DEVICE AND INTRAORAL X-RAY IMAGING SYSTEM USING SAME

(71) Applicants: Rayence Co., Ltd., Gyeonggi-do (KR); VATECH EWOO Holdings Co., Ltd., Gyeonggi-do (KR)

(72) Inventors: Yeong Kyun KIM, Gyeonggi-do (KR); Tae Woo KIM, Gyeonggi-do (KR)

(73) Assignees: Rayence Co., Ltd., Gyeonggi-do (KR); VATECH EWOO Holdings Co., Ltd., Gyeonggi-do (KR)

(21) Appl. No.: 15/557,077

(22) PCT Filed: Mar. 10, 2016
(86) PCT No.: PCT/KR2016/002404

§ 371 Date: Sep. 8, 2017

(30) Foreign Application Priority Data
Mar. 10, 2015 (KR) 10-2015-0033001

(51) Int. Cl. A61B 6/14; A61B 6/08

(52) U.S. Cl. CPC A61B 6/145; A61B 6/08

(57) ABSTRACT

The present invention provides an intraoral sensor device comprising: a sensor module containing a sensor panel; and a light-emitting device for radiating visible light or an infrared ray.

### TECHNICAL FIELD

The present invention relates to an intraoral sensor device and an intraoral X-ray imaging system using the same, and more particularly, to an intraoral sensor device and an intraoral X-ray imaging system, which enables the position at which the intraoral sensor device is inserted into the mouth to be determined easily and intuitively.

### BACKGROUND ART

In the conventional approach for intra-oral X-ray images to obtain X-ray images of teeth and surrounding tissues in the mouth, a film-based method is used.

In the case of using the conventional intraoral sensor device, it is difficult to determine where the intra-oral sensor device is located in the mouth, because there is no particular means of checking the position of the sensor device in the mouth.

This leads to a problem whereby a worker has to keep checking the position of the intraoral sensor device to place it in the correct position in the mouth of the patient, and whereby the patient has to endure the pain with his/her mouth open for a long while.

### DISCLOSURE

### Technical Problem

The present invention is to provide a scheme for easily identifying the position of an intraoral sensor device put in a mouth.

### Technical Solution

In accordance with an aspect of the present invention, disclosed is an intraoral sensor device including a sensor module, which includes a sensor panel, and a light-emitting device combined with the sensor module and emitting visible rays or infrared rays.

The light-emitting device may be arranged to correspond to at least one of the front or a side of the sensor module. The light-emitting device may be directly mounted on the sensor module. The intraoral sensor device may further include a cover that is equipped with the light-emitting device and is detachably attached to the sensor module.

In accordance with another aspect of the present invention, disclosed is a sensor-equipped device including: a first mounting portion having an intraoral sensor device mounted thereon; a second mounting portion having an X-ray generator mounted thereon to face the intraoral sensor device; a connector for connecting the first and second mounting portions; and a light-emitting device arranged in the first mounting portion and emitting visible rays or infrared rays.

The first mounting portion may include a first member for fixing the intraoral sensor device, and a second member for connecting the first member and the second mounting portion, and the light-emitting device may be mounted on at least one of the first member and the second member.

In accordance with another aspect of the present invention, disclosed is an intraoral imaging system including: an intraoral sensor device; a light-emitting device arranged in the intraoral sensor device, a cover that encloses at least a part of the intraoral sensor device, or a sensor-equipped device that is equipped with the intraoral sensor device and emits infrared rays; and an infrared sensor located outside the mouth for detecting the infrared rays.

In accordance with another aspect of the present invention, disclosed is an intraoral imaging system including: an intraoral sensor device; a light-emitting device arranged in the intraoral sensor device, or a cover that encloses at least a part of the intraoral sensor device, for emitting infrared rays; and an X-ray imaging device including an infrared sensor for detecting the infrared rays and generating information about the position of the intraoral sensor device, a projection module for projecting a sample image toward the intraoral sensor device based on the information, and an X-ray irradiator.

### Advantageous Effects

According to the present invention, a light-emitting device for emitting visible rays or infrared rays, which is a means of identifying the position of an intraoral sensor device, is combined with the intraoral sensor device or is mounted on a sensor-equipped device. Furthermore, an X-ray imaging device for detecting the position of the intraoral sensor device and projecting and displaying a sample image onto an outer surface of skin corresponding to the position is provided.

Accordingly, the position at which the intraoral sensor device is placed in the mouth may be easily identified from outside the mouth during an intraoral imaging. This may improve the convenience of the worker or the comfort of the patient during the intraoral imaging.

### DESCRIPTION OF DRAWINGS

FIGS. 1 to 3 are perspective, front, and side views, respectively, illustrating an intraoral sensor device according to a first embodiment of the present invention;

FIG. 4 is a side view illustrating an intraoral sensor device and an infrared sensor, according to the first embodiment of the present invention;

FIGS. 5 and 6 are plan views illustrating an intraoral sensor device according to a second embodiment of the present invention;

FIG. 7 is a plan view illustrating an intraoral sensor device according to a third embodiment of the present invention;

FIG. 8 is a plan view illustrating an intraoral sensor device according to a fourth embodiment of the present invention;

FIG. 9 is a perspective view illustrating an intraoral sensor device according to a fifth embodiment of the present invention;

FIG. 10 is a perspective view illustrating an intraoral sensor device and X-ray imaging device according to a sixth embodiment of the present invention; and

FIG. 11 is a perspective view illustrating a sensor-equipped device mounting an intraoral sensor according to a seventh embodiment.

### BEST MODE

Embodiments of the present invention will now be described in detail with reference to accompanying drawings.

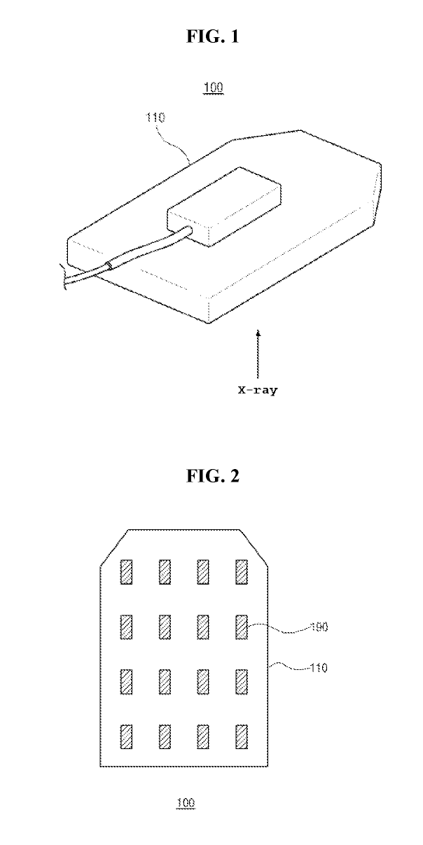

FIGS. 1 to 3 are perspective, front, and side views, respectively, schematically illustrating an intraoral sensor device according to a first embodiment of the present invention. FIG. 2 illustrates the front side of an intraoral sensor device, which is the side on which X-rays are incident.

Referring to FIGS. 1 to 3, an intraoral sensor device 100 in accordance with the first embodiment of the present invention may include a sensor module 110 and a light-emitting device 190.

Although not illustrated in detail, the sensor module 110 may include a sensor panel, for detecting X-rays and generating electrical signals, and a case that covers the exterior of the sensor panel.

In the sensor panel, there are photoelectric transducer elements for pixels arranged to form a matrix, which serve to detect light and generate electrical signals. The sensor panel may use a complementary metal-oxide semiconductor (CMOS) substrate or a thin film transistor (TFT) substrate, without being limited thereto.

The sensor panel may use a direct conversion method or an indirect conversion method. In the case of the direct conversion method, the photoelectric transducer element uses a photo conductor that reacts directly to incident X-rays to generate electrical signals.

On the other hand, in the case of the indirect conversion method, the sensor panel includes a scintillator for converting X-rays to visible rays, and the photoelectric transducer element is configured to respond the visible rays generated by the scintillator to generate electrical signals.

The case is arranged to protect the sensor panel by enclosing the entire exterior of the sensor panel.

On the outer side of the sensor module 110 having this configuration, at least one light-emitting device 190 may be mounted.

The light-emitting device 190 may emit visible rays or infrared rays. These visible rays or infrared rays may be particularly characterized in that they have a wavelength or intensity that enables them to penetrate human tissue, such as human skin.

In this way, with the visible rays or infrared rays that penetrate human tissue, when the intraoral sensor device 100 is inserted into the mouth, the visible rays or infrared rays generated by the light-emitting device 190 may penetrate the human tissue and may be identified externally.

When visible rays are output from the light-emitting device 190, they may be immediately identified with the naked eye.

On the other hand, when infrared rays are output from the light-emitting device 190, they may be identified by a separate infrared detection device, such as an infrared sensor 180 outside the mouth. The infrared sensor 180 and the intraoral sensor device 100 may constitute an intraoral X-ray imaging system. For example, the infrared sensor 180 may be mounted or attached onto an X-ray imaging device that radiates X-rays to the intraoral sensor device 100 from outside the mouth for an intraoral X-ray image. The intraoral X-ray imaging system will be described in more detail later. Furthermore, although not shown, if visible rays are output from the light-emitting device 190, they may be identified using a photo sensor located outside the mouth.

As such, the position of the intraoral sensor device 100 may be easily figured out through light emitted from the light-emitting device 190.

In the meantime, as the light-emitting device 190, a light emitting diode (LED) for emitting visible rays or infrared rays may be used, without being limited thereto. Furthermore, the light-emitting device 190 may emit visible rays or infrared rays having at least one different wavelength band, at least one emission pattern of at least one different on/off period, and at least one different shape, and the photo sensor or infrared sensor may detect the wavelength, the emission pattern, or the shape of the sensed visible rays or infrared rays. Accordingly, even with the detection results of only some of the light-emitting devices 190, the position of the intraoral sensor may be estimated more accurately measured as a result of detecting the visible rays or infrared rays.

In the meantime, when it comes to the arrangement of the light-emitting device 190, taking into account the aspect of light penetration to the outside of the mouth, the light-emitting device 190 may be arranged in at least one of the front (i.e. the side on which X-rays are incident) and the sides of the sensor module 110. In the case of the infrared-based light-emitting device 190, the light-emitting device 190 may be arranged in the rear side (i.e. the side opposite the side on which X-rays are incident) of the sensor module 110.

In this regard, as shown in FIGS. 2 and 3, the light-emitting device 190 may be arranged in a part or all of the front of the sensor module 110 to emit visible rays or infrared rays. Although FIGS. 2 and 3 illustrate the light-emitting device 190 arranged across the entire front of the sensor module 110, the arrangement of the light-emitting device 190 is not limited thereto, and the light-emitting device 190 may be arranged in part of the front of the sensor module 110 in various forms.

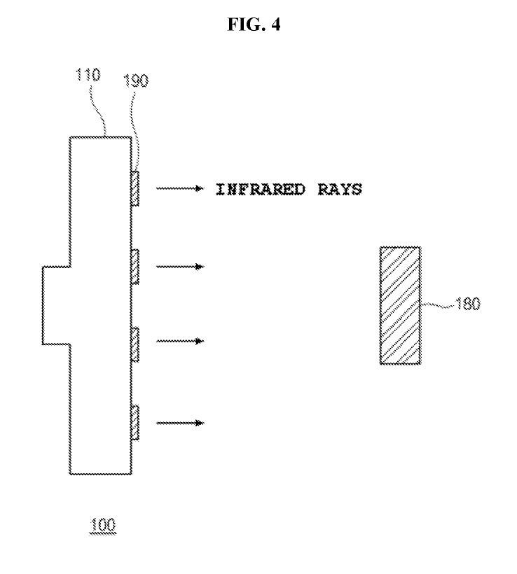

Alternatively, in a second embodiment shown in FIGS. 5 and 6, the light-emitting device 190 may be arranged on the sides of the sensor module 110. FIG. 5 illustrates the light-emitting device 190 arranged along the sides of the sensor module 110, and FIG. 6 illustrates the light-emitting device 190 arranged at corners of the sides of the sensor module 110.

In this way, the light-emitting device 190 may be arranged in at least some of the sides of the sensor module 110 in various forms.

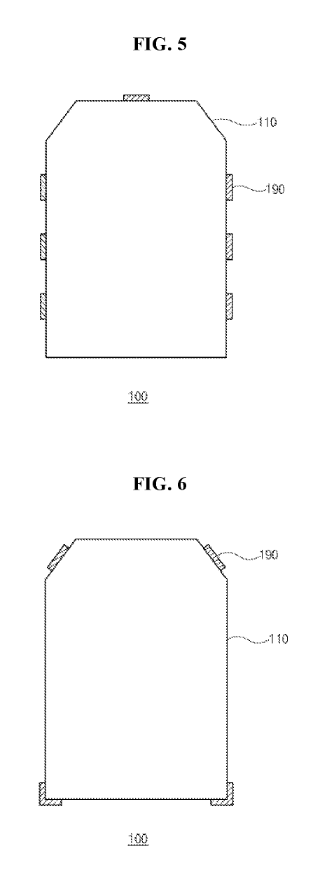

FIG. 7 is a plan view schematically illustrating an intraoral sensor device according to a third embodiment of the present invention.

The intraoral sensor device 100 in accordance with the third embodiment of the present invention may include a sensor module 110 and a cover 200, which is detachably attached to the sensor module 110 and has light-emitting devices 190 arranged therein.

The cover 200 is flexible, and is formed to be detachably attached to the sensor module 110, for example, as shown in FIG. 7, and the cover 200 may be formed to enclose at least one outer side of the sensor module 110 and may define an opening 201 that exposes the top and bottom of the sensor module 110. In FIG. 7, the cover 200 encloses a part of the outer periphery of the sensor module 110 and is formed as a frame having one side open when viewed in plan.

The cover 200 may be formed to have sides, exclusive of the open side, indented such that the cross-section of the cover, which is perpendicular to the length direction, wraps the corresponding exterior of the sensor module 100, allowing the sensor module 110 to be inserted into the indentation in the cover 200 and to be combined with the cover 200.

In at least a portion of the cover 200, the light-emitting device 190 may be mounted. In this case, the cover 200 may be formed to have a property of light transmittance, meaning that it is transparent to the light generated by the light-emitting device 190. For example, if the light-emitting device 190 emits visible rays, the cover 200 may be formed of a material having high transmittance to visible rays; if the light-emitting device 190 emits infrared rays, the cover 200 may be formed of a material having high transmittance to infrared rays.

In this way, with the light-emitting device 190 arranged in the cover 200, when the intraoral sensor device 100 having the cover 200 combined with the sensor module 110 is inserted into the mouth, visible rays or infrared rays generated by the light-emitting device 190 may penetrate the human tissue and may be checked externally.

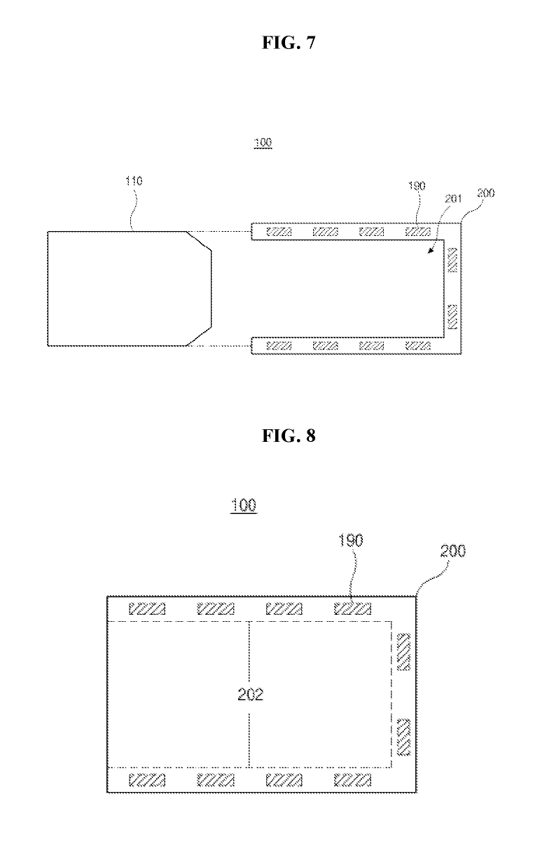

In another example of the cover 200, as shown in FIG. 8, the cover 200 may be formed to cover not only the outer sides but also the top and bottom of the sensor module 110. Specifically, the cover 200 of FIG. 8 has a form that covers both the top and bottom of an opening 201, and may be shaped like a pocket with one side having an inlet into which the sensor module 110 is inserted, thereby defining a receiving space 202 for mounting the sensor module 110.

The light-emitting device 190 may also be arranged in at least a portion of the cover 200 of FIG. 8. The light-emitting device 190 may be arranged to correspond to the front or side of the sensor panel 110, without being limited thereto. In the meantime, similar to FIG. 7, FIG. 8 illustrates the light-emitting device 190 arranged in the sides of the sensor panel 110.

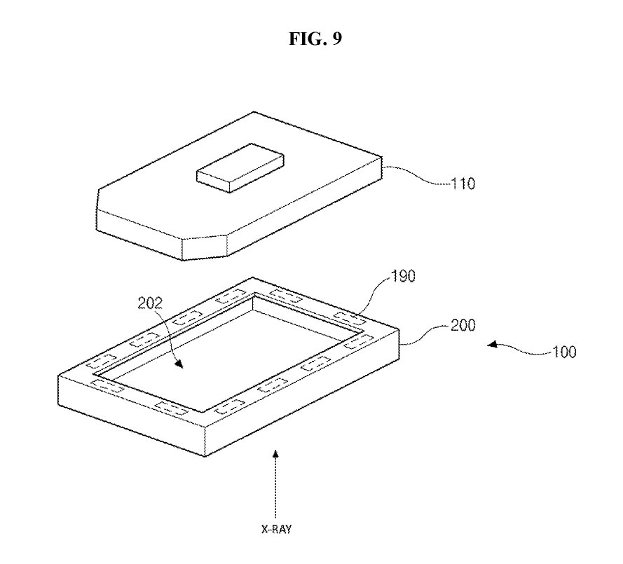

In still another example of the cover 200 of the present invention, as shown in FIG. 9, the cover 200 may be formed to cover not only the outer sides but also the entire bottom (i.e. the side on which X-rays are incident) of the sensor module 110 and cover the edges of the top (i.e. the side opposite the side on which X-rays are incident). Accordingly, the cover 200 may define the receiving space 202 for receiving the sensor module 110.

The light-emitting device 190 may also be arranged on at least a part of the cover 200 of FIG. 9. The light-emitting device 190 may be arranged to correspond to the front or side of the sensor panel 110. In the meantime, similar to FIG. 8, FIG. 9 illustrates the light-emitting device 190 arranged in the sides of the sensor panel 110.

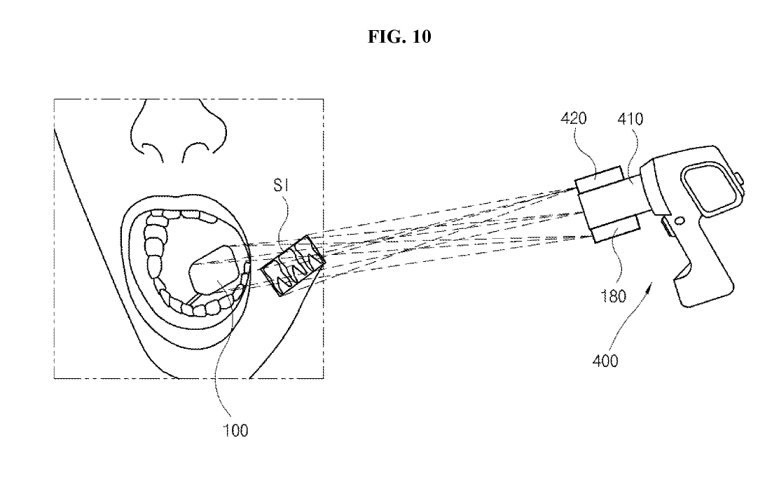

FIG. 10 schematically illustrates an intraoral X-ray imaging system including an intraoral sensor device and an X-ray imaging device, according to a sixth embodiment of the present invention, and schematically illustrates the taking of an X-ray image.

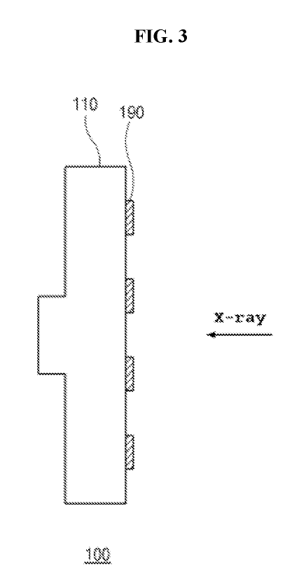

Referring to FIG. 10, as the intraoral sensor device 100 of the present embodiment, the aforementioned intraoral sensor device equipped with the light-emitting device 190 (see FIGS. 2 to 9) for outputting infrared rays may be used.

An X-ray imaging device 400 includes an X-ray irradiator 410 for generating X-rays and radiating them on the intraoral sensor device 100. The X-ray irradiator 410 is located on the front side of the X-ray imaging device 400.

Furthermore, the X-ray imaging device 400 may include an infrared sensor 180 for detecting infrared rays emitted from the emitting device of the intraoral sensor device 100. The infrared sensor 180 may be located on the front part of the X-ray imaging device 400, for example, on the X-ray irradiator 410, without being limited thereto.

Furthermore, the X-ray imaging device 400 may include a projection module 420 that projects a sample image SI, which is an image used for the purpose of checking with the naked eye to more easily and accurately identify the position at which the intraoral sensor device 100 is inserted into the mouth. The projection module 420 may be located on the front part of the X-ray imaging device 400, e.g., on the X-ray irradiator 410, such that the direction of image projection substantially corresponds to the direction of X-ray radiation of the X-ray irradiator 410.

An X-ray imaging process using the X-ray imaging device 400 will now be examined.

First, the infrared sensor 180 detects infrared rays emitted from the emitting device of the intraoral sensor device 100 and generates information about the position of the intraoral sensor device 100. In this regard, preferably, a separate operation processing unit for detecting the sensing results of the infrared sensor 180 and calculating the information about the position of the intraoral sensor device 100 may be included, in which case the light-emitting device 190 may emit infrared rays having different wavelengths, emit patterns, or shapes, and the operation processing unit may calculate more accurate information about the position of the intraoral sensor device 100 using infrared rays even from only some light-emitting devices 190 by comparing the relative positional relationship between each light-emitting devices 190 and the intraoral sensor 100, which is stored in advance, with the result of sensing by the infrared sensor 180.

The information about the position, produced by the infrared sensor 180, is delivered to the projection module 420, and in response, the projection module 420 projects the sample image SI toward where the intraoral sensor device 100 is, to present the sample image SI on the outer surface of the skin in the projection direction. Any type of image that may be identified with the naked eye may be used for the sample image SI. Preferably, a separate operation processing unit for calculating the approximate position of the intraoral tissue to be currently imaged, such as a front tooth, a molar tooth, an occlusion, etc., based on the position of the intraoral sensor device 100 and/or the inclination angle of the X-ray imaging device 400, in which case the operation processing unit may also select a sample image SI that corresponds to the position in the mouth to be currently imaged, from among sample images SIs stored in advance for respective positions, such that the selected sample image SI is projected using the projection module 420.

A worker may then easily check the position of the intraoral sensor device 100 placed in the mouth with his/her naked eye using the sample image SI projected on the surface of the skin. Accordingly, the person may accurately place the intraoral sensor device 100 at a desired position while checking the sample image SI, and may then operate the X-ray imaging device 400 to complete an X-ray image.

As such, in the present invention, projecting a sample image on the surface of the skin corresponding to the position of the intraoral sensor device 100, detected using infrared rays, may ensure that the position of the intraoral sensor device 100 put in the mouth may be identified with the naked eye, and accordingly, an X-ray image of a desired intraoral part may be effectively performed.

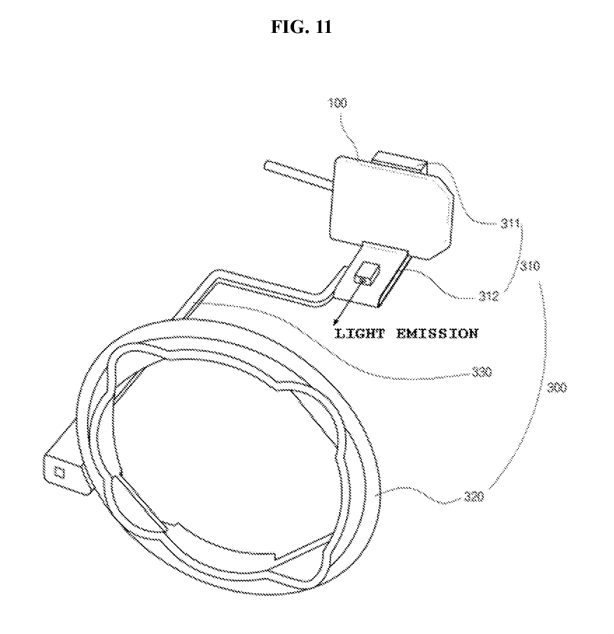

FIG. 11 is a perspective view schematically illustrating a sensor-equipped device having an intraoral sensor mounted thereon, according to a seventh embodiment of the present invention. The sensor-equipped device may be included in the intraoral X-ray imaging system in accordance with the present invention.

Referring to FIG. 11, the sensor-equipped device 300 in accordance with the present embodiment corresponds to a tool for supporting intraoral images by helping the intraoral sensor device 100 and the X-ray imaging device 400 (see FIG. 10) face each other in a straight line, and is also referred to as a so-called “cone indicator” or “extension cone paralleling”. The X-ray imaging device may include the X-ray irradiator 410 (see FIG. 10), and may occasionally include, similar to the sixth embodiment, the infrared sensor 180 and/or the projection module 420.

The sensor-equipped device 300 may be mounted in the intraoral sensor device 100, and may include a first mounting portion 310, on which the intraoral sensor device 100 is mounted and the light-emitting device 190 is arranged, a second mounting portion 320, on which the X-ray imaging device, arranged to face the intraoral sensor device 100 for irradiating X-rays, is mounted, and a connector 330 for connecting the first and second mounting portion 310, 320.

The first mounting portion 210, on which the intraoral sensor device 100 is mounted, may include a first member 311 for holding and fixing the intraoral sensor device 100, and a second member 312 connected to one side of the first member 311 and extending forward of the intraoral sensor device 100 to connect the first member 311 and the connector 330. The second member may be a bit that a person to be imaged bites.

The light-emitting device 190 for emitting visible rays or infrared rays may be arranged in one side of the first and/or second member 311, 312. Specifically, the light-emitting device 190 may be mounted at a position that is not hidden by the intraoral sensor device 100, and, for example, may be located closer to the X-ray generator than the intraoral sensor device 100 is.

With the light-emitting device 190 arranged in the sensor-equipped device 300, the visible rays or infrared rays generated by the light-emitting device 190 may penetrate the human tissue and may be checked externally.

As described above, according to the present invention, the light-emitting device for emitting visible rays or infrared rays, which is a means of identifying the position of the intraoral sensor device, is combined with the intraoral sensor device or is mounted in the sensor-equipped device.

Accordingly, the position where the intraoral sensor device is put in the mouth may be externally and easily identified during an intraoral imaging. This may improve the convenience of the worker and the comfort of the patient during the intraoral imaging.

There may be a single light-emitting device 190 or a plurality of light-emitting devices 190, and unlike what is shown in the drawings, the light-emitting device 190 may have a shape or arrangement that allows identification of up, down, left, or right positions, such as a triangle, a rectangle, a cross, etc. This may enable the position, angle, etc., of the intraoral sensor device placed in the mouth to be more easily identified from outside the mouth.

1. An intraoral sensor device comprising:
a sensor module including a sensor panel;
a cover attached to the sensor module; and
a light-emitting device mounted in the cover and emitting visible rays or infrared rays.

2. The intraoral sensor device of claim 1, wherein the light-emitting device is arranged in at least one of a front or a side of the sensor module.

3. (canceled)

4. The intraoral sensor device of claim 1, wherein the cover is detachably attached to the sensor module.

5. (canceled)

6. (canceled)

7. (canceled)

8. An intraoral imaging system comprising:
an intraoral sensor device;
a light-emitting device lighting infrared rays mounted in the intraoral sensor device, a cover enclosing at least a part of the intraoral sensor device; and
a X-ray imaging device including an infrared sensor generating position data of the intraoral sensor device by detecting the infrared rays, a projection module projecting a sample image toward the intraoral sensor device, and an X-ray irradiator.

9. The intraoral sensor device of claim 1, wherein the cover has an inlet for inserting the sensor module and a receiving space for mounting the sensor module.

10. The intraoral sensor device of claim 1, wherein the cover is enclosing the periphery of the sensor module and is formed as a frame having one side open.

11. The intraoral sensor device of claim 1, wherein the light-emitting device emits the visible rays or the infrared rays in different wavelength bands.

12. The intraoral sensor device of claim 1, wherein the light-emitting device emits the visible rays or the infrared rays in different light emission patterns of different on/off periods.

13. The intraoral sensor device of claim 1, wherein the light-emitting device emits the visible rays or the infrared rays in a predetermined shape.

14. The intraoral sensor device of claim 13, wherein the light-emitting device emits the visible rays and the infrared rays in at least two different predetermined shapes.

15. The intraoral imaging system of claim 8, wherein the cover has an inlet for inserting the sensor module and a receiving space for mounting the sensor module.

16. The intraoral imaging system of claim 8, wherein the cover is enclosing the periphery of the sensor module and is formed as a frame having one side open.

17. The intraoral imaging system of claim 8, wherein the light-emitting device emits the infrared rays in different wavelength bands and the infrared sensor detects the different wavelength bands.

18. The intraoral imaging system of claim 8, wherein the light-emitting device emits the infrared rays in different light emission patterns of different on/off periods.
